(19) United States
(12) Patent Application Publication
(10) Pub. No.: US 2012/0302834 A1
(43) Pub. Date: Nov. 29, 2012

(54) COVER FOR A LAPAROSCOPE, SYSTEMS INCLUDING THE COVER AND METHODS OF USE

(75) Inventors: Michael White, Leeds (GB); Michael John McMahon, Leeds (GB)

(73) Assignee: SURGICAL INNOVATIONS LIMITED, Leeds, Yorkshire (UK)

(21) Appl. No.: 13/576,910

(22) PCT Filed: Feb. 2, 2011
(86) PCT No.: PCT/GB11/50175

§ 371 Date: Aug. 2, 2012

(30) Foreign Application Priority Data
Feb. 4, 2010 (GB) 1001812.5

(51) Int. Cl. A61B 1/313; G01M 3/04

(52) U.S. Cl. 600/124; 600/121; 73/40

(57) ABSTRACT

A cover for a laparoscope having a substantially gas-impermeable, flexible membrane defining an interior space adapted to receive at least a distal-most portion of the laparoscope; and at least one valve for allowing gas to flow into and/or out of the interior space. By providing a substantially gas-impermeable, flexible membrane, the membrane can conform to the camera in use, ensuring that the cover can be used with both rigid and flexible laparoscopes and in particular those with cameras at their distal end. The valve allows gas to flow into or out of the interior space. This may be used to remove gas from the interior space so that the cover conforms more closely to the profile of the endoscopic camera. It may also be used to check that there has been no damage to the cover during the operation, and thus no breach of the sterile barrier.

The present invention relates to a sterile sheath, cover or drape for a surgical instrument and in particular to a cover, sheath or drape for a flexible endoscopic camera or camera holding system, known as a flexible laparoscope. Other aspects of the invention also relate to methods and systems for applying the cover to an endoscopic camera or laparoscope and for checking the cover for leaks.

Most minimally invasive surgical procedures require use of an endoscopic camera during the procedure. In conventional minimally invasive surgery the endoscopic camera is attached to the exterior portion of a rigid endoscope usually referred to as a laparoscope, although other names such as arthroscope are used for special applications. More recently combined endoscope and camera systems have been developed such that the charged couple device (CCD) is situated at the internal extremity of the instrument, and is placed in front of a flexible part of the instrument. This enables the operator to flex the tip of the instrument that bears the objective lens, the source of illumination and the CCD, so that an oblique or even right-angled, view of the operative area can be obtained. Because they incorporate the CCD, a lens system and the illumination in a single instrument, these endoscopic instruments are much more expensive than the conventional laparoscopes. Traditionally, the laparoscope has been re-sterilised, but there has been the option to re-use the camera head (the more expensive part of the combination) by enclosing it in a sterile drape. While this is advantageous, these provide no easy way to guarantee the integrity of the drape, and hence the sterility of the camera head before or after use.

Many hospitals have now outsourced their decontamination and sterilisation of surgical instruments. This means that there is a greater delay than when the sterilisation and decontamination facilities are provided on site. For example, with an onsite facility an instrument could be decontaminated and sterilised for reuse within two hours. With an outsourced department, it is likely to be around three days before the instrument is received back and available for reuse.

Depending on the particular procedure, a surgeon may be able to carry out up to 6 or 8 operations in a day. With the extra delay involved in outsourcing sterilisation and decontamination, this requires the hospital to hold a greater inventory of instruments in order to allow a surgeon to have sufficient to carry out procedures throughout the day. When the instruments have a high capital cost, like endoscopic cameras, the increase in inventory and capital cost reduces the savings from outsourcing the sterilisation and decontamination of instruments.

Accordingly, the present invention provides a cover, sheath or drape for a surgical instrument that surrounds the surgical instrument in use, protecting the instrument against contamination in the operating theatre. The invention includes means, such as a valve or tap, for evacuating and/or inflating the cover when it is in place on an instrument. This can allow the cover to be checked for leaks and its integrity confirmed before and/or after a procedure. After use, the cover can be removed from the instrument and disposed of. There is no need to decontaminate and re-sterilise the instrument before it is used in another surgical procedure if no leak in the cover has been detected.

According to a first aspect of the present invention, there is provided cover for a laparoscope, the cover comprising a substantially gas-impermeable, flexible membrane defining an interior space adapted to receive at least a distal-most portion of the laparoscope; and at least one valve for allowing gas to flow into and/or out of the interior space.

The laparoscope may be any minimally invasive surgical instrument and preferably includes a camera system, such as a CCD and light source, at its distal end. The laparoscope may be an endoscopic camera, that is a combination of an endoscope and a camera system. The term “cover” is used to refer to any item that can function as a cover, and includes sheaths and drapes. The term “valve” is used to refer to any item that can control the flow of gas and thus also includes taps.

The flexible membrane must cover the camera sufficiently such that it is protected against contamination during the surgical procedure. Thus, it must at least cover the distal-most portion which will be inserted into the patient during the procedure, although more preferably the cover is adapted to cover substantially all, or all, of the endoscopic camera.

By providing a substantially gas-impermeable, flexible membrane, the membrane can conform to the camera in use, ensuring that the cover can be used with both rigid and flexible laparoscopes and in particular those with cameras at their distal end. The valve allows gas to flow into or out of the interior space. This may be used to remove gas from the interior space so that the cover conforms more closely to the profile of the endoscopic camera. It may also be used to check that there has been no damage to the cover during the operation, and thus no breach of the sterile barrier. This is discussed in more detail below.

In some embodiments, the cover may further comprise means for creating a gas-tight seal around at least a portion of the laparoscope. For example the flexibility of the cover itself may allow it to conform closely to the laparoscope and form a gas tight seal. Alternatively a specific seal may be provided at a proximal end, such as an adjustable or resilient seal for sealing onto the surface of the laparoscope. When used with an endoscope camera, a gas-tight seal around the camera body or the connecting cable may be provided that enables the system to be evacuated so that there is a contact fit between the components of the cover, sheath or drape and the flexible laparoscope. At the completion of the procedure, or during the procedure if there are concerns about the continuing integrity of the sterile barrier, the cover, sheath or drape may be inflated to check for air leaks; a leak indicating that the sterile barrier has been compromised.

The cover can be made from a variety of materials, for example latex, polyethylene or polyurethane. Different parts of the cover may require materials with different specifications. Preferably the membrane is optically clear, or when used with an endoscopic camera has at least an optically clear portion adapted to be positioned next to the camera lens in use. This ensures that the membrane does not interfere with the imaging of the camera.

In some embodiments, the distal end of the cover may include a transparent disc adapted to be adjacent to the lens of an endoscopic camera. The transparent disc may be made of a more rigid material than the rest of the cover, for example it may be made from polycarbonate. This allows uninterrupted vision through the lens of an endoscopic camera. Proximal to this the cover is preferably thin to minimally interfere with the passage of the instrument through the cannula, and sufficiently elastic that the variable curvature of the endoscope can be accommodated. This is important in minimally invasive procedures where space is at a premium.

In one embodiment the cover may comprise a single valve that can be actuated to allow gas flow into or out of the interior space. In one embodiment such a valve has a Luer connection, for example to allow air to be evacuated or introduced using a syringe.

In another embodiment the cover comprises a first one-way valve configured to allow gas to flow into but not out of the interior space and a second one-way valve configured to allow gas to flow out of but not in to the interior space. This arrangement is less preferred as it adds to the bulk of the cover.

The substantially gas-impermeable, flexible membrane may be configured to allow operation of controls on a laparoscope, when the cover is in use. This may include providing means for allowing adjustment of the focus, zoom and/or both left to right and up and down movement of the flexible part of an endoscope camera through the cover. For example the cover may comprise extra material in the areas of controls on the laparoscope, such as pouches shaped to accommodate the adjustment controls under the cover and positioned for use with the controls on specific endoscopic cameras. Such pouches are preferably produced in a thin and easily deformable material that can be contracted down to adopt the shape of the control, and allow its full and free range of movement when the cover is in place and has been evacuated.

The cover of the present invention can be provided as part of a system for applying a cover to a laparoscope. The laparoscope may be a flexible endoscope that incorporates a camera. Such a system also includes a vacuum means for removing gas present in the interior space of the cover such that the flexible membrane conforms to the shape of the endoscopic camera. The vacuum means may be any suitable device for sucking gas or air from the interior space such as a syringe, a vacuum pump or any other negative pressure source. By removing gas from the cover it conforms very closely to the shape of the laparoscope and adds minimal bulk to the instrument. This allows the instrument to be used more easily with the cover having minimal effect on the operation of the laparoscope.

In another aspect of the invention, a system for checking a cover for leaks is provided. The system comprises a cover and inflation means for inflating the cover. This allows the cover to be easily checked for leaks. The cover is substantially gas impermeable and therefore inflating the cover will allow easy detection of leaks. If a leak is detected, the cover can be discarded since the presence of a leak indicates a hole through which the laparoscope might be contaminated, preventing its reuse.

In a further aspect of the invention, the system can include both vacuum means and inflation means allowing the same system to check a cover for leaks and apply it to the endoscopic camera. In this case, the vacuum means and inflation means could be combined into a single device that can both create a vacuum and inflate the cover when required.

Another embodiment of the invention comprises a gas-tight seal between the cover and the proximal (external) part of the laparoscope or its connecting cable such that the cover around the distal (internal) part of the endoscope can be evacuated or inflated. This seal may incorporate valves, elasticity of the material of the cover, or an external wrapping or binding mechanism. The cover may be contiguous with a drape that will itself provide a sterile cover for the cable or other connecting link between the endoscope and the light source, insufflators and camera box.

The systems of the present invention can be used in a method of applying a cover to a laparoscope, such as an endoscope incorporating a CCD, comprising:
providing a system as discussed above and a laparoscope;
inserting the laparoscope into the interior space of the cover;
connecting vacuum means to a valve of the cover;
operating the vacuum means to remove gas from the interior space of the cover, such that the flexible membrane of the cover conforms to the shape of the laparoscope; and
disconnecting the vacuum means when the cover has substantially conformed to the shape of the laparoscope.

The systems can also be used in a method for checking a cover for a laparoscope for leaks comprising:
providing a system as discussed above;
connecting the inflation means to the valve of the cover;
inflating the cover using the inflation means;
checking the cover for gas leaks while inflated;

The inflation means could be controlled so as to ensure that a maximum pressure is not exceeded within the cover during the inflating of the cover. This can prevent bursting of the cover. The checking the cover for leaks could be carried out by several means, for example, visual or audio. Some methods could include imaging the cover in the inflated state to establish leaks, or checking audibly for any hissing noises which indicate leaks. The cover can be checked for leaks without an endoscopic camera present, or after an endoscopic camera has been placed within the cover.

Embodiments of the invention will now be described by way of example with reference to the accompanying drawings, in which:

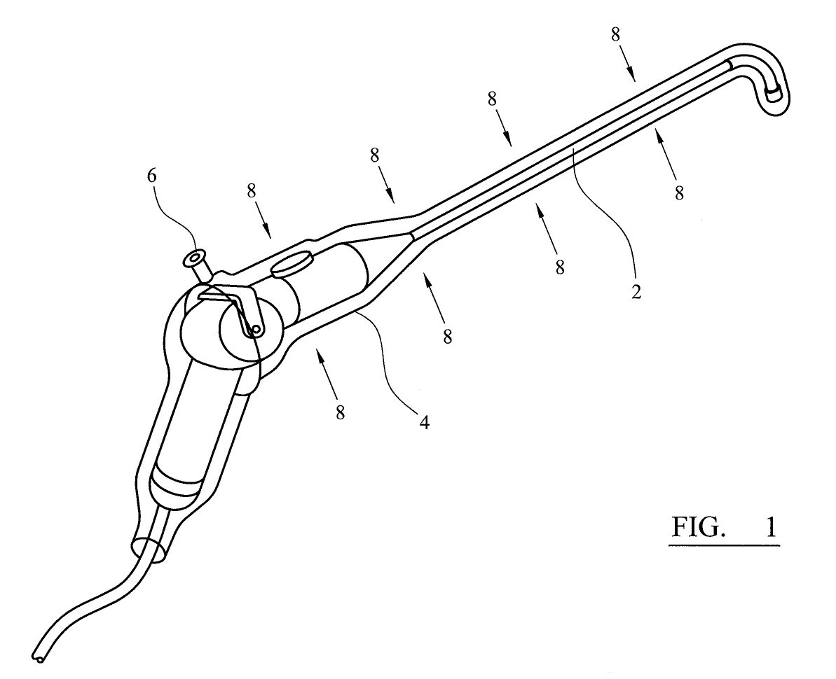

FIG. 1 is a diagrammatic representation of a cover according to an embodiment of the present invention applied to a flexible endoscope that incorporates a CCD;

FIG. 2 is a diagrammatic representation of a perspective view of cover according to another embodiment of the invention applied to a flexible endoscope that incorporates a CCD;

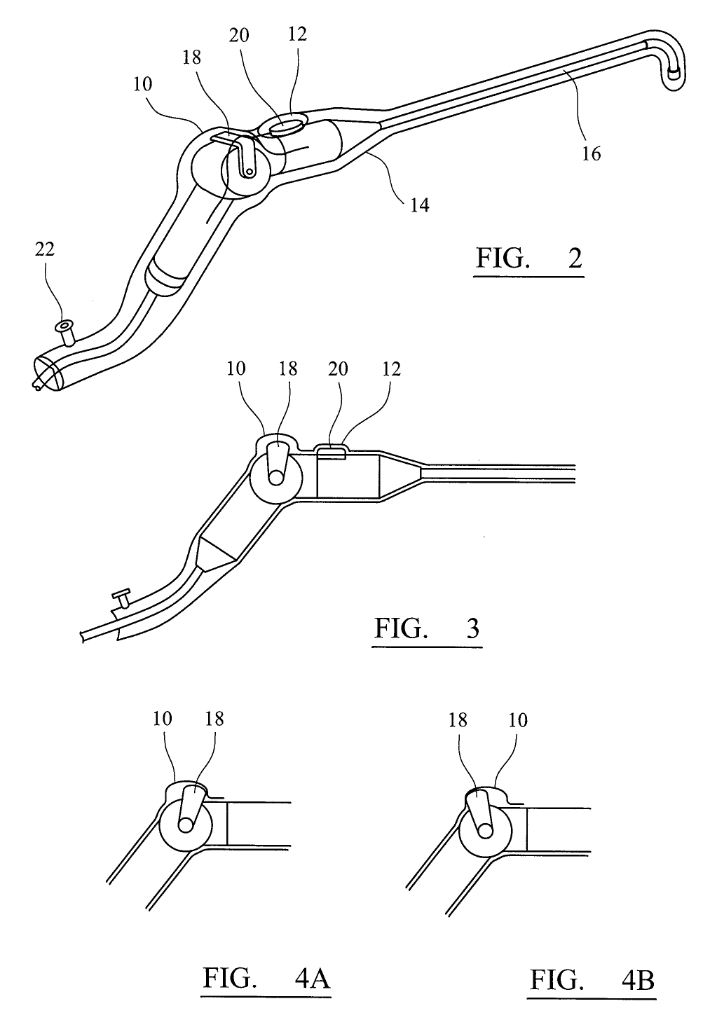

FIG. 3 is a diagrammatic representation of a side view of part of the embodiment of FIG. 2;

FIGS. 4A and 4B are diagrammatic representations showing detail of the embodiment of FIG. 2.

FIG. 1 depicts a cover according to a first embodiment of the present invention applied to an endoscopic camera 2. In this embodiment the endoscopic camera 2 is a flexible endoscopic camera but the invention can equally be applied to a rigid camera. The cover according to the embodiment of the invention comprises a gas-impermeable, flexible membrane 4 which defines an interior space into which the endoscopic camera 2 has been inserted. As depicted in FIG. 1, the membrane 4 is optically clear, allowing the endoscopic camera 2 to be seen through the membrane 4. In alternate embodiments only the distal portion is optically clear or transparent, the remainder can be semi-transparent or opaque depending on whether it is necessary to see through the cover, for example to a control.

The cover also comprises a Luer connection 6 with a tap or valve so that vacuum can be maintained during the use of the system in surgery. The Luer connection 6 can be connected to either a vacuum pump, a syringe or a pressure source, such as a gas supply. To apply the cover to the endoscopic camera the endoscopic camera is first inserted into the interior space defined by the flexible membrane 4. This could be achieved in a variety of ways, for example rolling the cover 4 over the camera 2, or simply inserting the camera 2 into the cover 4.

The cover optionally includes a separate gas-tight seal at its proximal end (not shown). Alternatively, the gas-tight seal can be provided by the flexibility of the cover itself.

In used, the endoscope is held by the non-sterile assistant and the cover by the scrub nurse. Once the endoscope has been inserted into the cover the scrub nurse will advance the cover to cover the connecting cable, and will secure the gas tight seal if one is present. Once the camera has been inserted a syringe or vacuum pump (not shown) is connected to the Luer connector 6 and used to remove any gas or air present between the camera and the membrane 4. This causes the membrane 4 to closely adhere to the shape of the endoscopic camera 2 under atmospheric pressure, as indicated by arrows 8 in FIG. 1. The vacuum is maintained using a tap or one-way valve.

After use, the valve or tap can be actuated to allow air or gas to reenter the cover and the cover can be removed.

Optionally, the cover of the present invention can be checked for leaks, this can occur both before and/or after a surgical procedure. In order to check the cover for leaks an inflation means, such as a syringe or pump (not shown) is attached to connector 6 and used to inflate the cover 4. Any leaks in the membrane 4 can be detected by, for example, visual or audible means such as looking for visible tears or listening for hissing noise of gas escaping or by immersion in liquid.

In a preferable embodiment the cover is checked for leaks both before and after a procedure in order to ensure its integrity at the beginning and at the end. The sterile nature of the endoscopic camera can then be assured.

Another embodiment of the cover of the present invention, applied to an endoscopic camera, is depicted in FIG. 2. The construction of this embodiment is the same as the embodiment of FIG. 1, except as described below.

This cover 14 depicted in FIG. 2 includes pouches 10 and 12. The pouches 10 and 12 contain additional flexible material and are positioned so that when the cover 14 is installed on the endoscopic camera 16 there is more freedom for a user to operate controls 18 and 20 of the endoscopic camera 16. The pouches 10 and 12 can enable easier operation of controls through the cover 14 than the embodiment of FIG. 1. The valve 22 has also been positioned on the cover 14 so that it does not interfere with operation of the controls 18 and 20.

A side view of the embodiment of FIG. 2 is depicted in FIG. 3. FIGS. 4A and 4B show how the pouch 10 assists in the movement of control 18 between first and second positions.

In use, when the cover 16 is applied to an instrument and evacuated, the cover 16 will conform closely to the surface of the instrument. FIGS. 2, 3, 4A and 4B exaggerate the pouches 10 and 12 so that their function can be understood more clearly. In use, the extra material may conform more closely to the instrument than is depicted, but it will still assist in movement of the controls.

Thus, the present invention provides a cover which allows an endoscopic camera to be maintained in sterile conditions during a surgical procedure. This allows the endoscopic camera to be reused without requiring sterilisation. It allows a hospital to hold a smaller inventory than would be required to account for necessary delays while surgical instruments are decontaminated and sterilised before they are used in subsequent procedures.

1. A cover for a laparoscope, the cover comprising:
a substantially gas-impermeable, flexible membrane defining an interior space adapted to receive at least a distal-most portion of a laparoscope; and
at least one valve for allowing gas to flow into and/or out of the interior space.

2. A cover according to claim 1, further comprising means for creating a gas-tight seal around at least a portion of the laparoscope.

3. A cover according to claim 1, which comprises a single valve to allow gas to flow both into and out of the interior space.

4. A cover according to claim 1, which comprises a first one-way valve configured to allow gas to flow into but not out of the interior space and a second one-way valve configured to allow gas to flow out of but not in to the interior space.

5. A cover according to claim 1, wherein the substantially gas-impermeable, flexible membrane is configured to allow operation of controls on a laparoscope, when the cover is in use.

6. A system for checking a cover for a laparoscope for leaks, the system comprising:
a cover according to claim 1; and
inflation means adapted to be attached to the valve for inflating the cover.

7. A system for applying a cover to a laparoscope, the system comprising:
a cover according to claim 1; and
vacuum means for removing gas present in the interior space of the cover such that the flexible membrane conforms to the shape of the laparoscope.

8. A system for applying a cover to a laparoscope and for checking the cover for leaks, the system comprising:
a cover according to claim 1;
vacuum means for removing gas present in the interior space of the cover such that the flexible membrane conforms to the shape of the laparoscope; and
inflation means adapted to be attached to the valve for inflating the cover.

9. A method for checking a cover for a laparoscope for leaks, the method comprising:
providing a system according to claim 6;
connecting the inflation means to the valve of the cover;
inflating the cover using the inflation means;
checking the cover for gas leaks while inflated;

10. A method according to claim 9, further comprising:
providing an laparoscope;
inserting the laparoscope into the interior space of the cover before inflating the cover.

11. A method of applying a cover to a laparoscope, the method comprising:
providing a system according to claim 7 and a laparoscope;
inserting the laparoscope into the interior space of the cover;
connecting vacuum means to a valve of the cover;
operating the vacuum means to remove gas from the interior space of the cover, such that the flexible membrane of the cover conforms to the shape of the laparoscope; and
disconnecting the vacuum means when the cover has substantially conformed to the shape of the laparoscope.

12. A method for checking a cover for a laparoscope for leaks, the method comprising:
providing a system according to claim 8;
connecting the inflation means to the valve of the cover;
inflating the cover using the inflation means;
checking the cover for gas leaks while inflated;

13. A method according to claim 12, further comprising:
providing an laparoscope;
inserting the laparoscope into the interior space of the cover before inflating the cover.

14. A method of applying a cover to a laparoscope, the method comprising:
providing a system according to claim 8 and a laparoscope;
inserting the laparoscope into the interior space of the cover;
connecting vacuum means to a valve of the cover;
operating the vacuum means to remove gas from the interior space of the cover, such that the flexible membrane of the cover conforms to the shape of the laparoscope; and
disconnecting the vacuum means when the cover has substantially conformed to the shape of the laparoscope.
